(19) United States
(12) Patent Application Publication
(10) Pub. No.: US 2006/0216820 A1
(43) Pub. Date: Sep. 28, 2006

(54) Use of retinoic esters of hyaluronic acid for the differentiation of totipotent stem cells

(75) Inventors: Alberto Perbellini, Verona (IT); Carlo Ventura, Bologna (IT); Margherita Maioli, Sassari (IT)

Correspondence Address: OHLANDT, GREELEY, RUGGIERO & PERLE, LLP, ONE LANDMARK SQUARE, 10TH FLOOR, STAMFORD, CT, 06901, US

(73) Assignee: SINTOFARM S.P.A., Guastalla (IT)

(21) Appl. No.: 10/542,302

(22) PCT Filed: Jan. 14, 2004
(86) PCT No.: PCT/EP04/00183

§ 371 Date: Jul. 14, 2005

(30) Foreign Application Priority Data
Jan. 14, 2003 (IT) MI2003 A 000043

(51) Int. Cl. A61K 31/728; C12N 5/08; C12N 5/04; C08B 37/00; C12N 5/06

(52) U.S. Cl. 435/325; 435/366; 514/54; 536/53; 435/353; 435/354; 435/358; 435/362

(57) ABSTRACT

The present the invention relates to a new use of retinoic of hyaluronic acid, that exhibit pre-differentiating activity on totipotent stem cells. The invention also relates to a process to differentiate said stem cells and to select molecules capable of modulating the pro-differentiating activity of those esters.

### FIELD OF THE INVENTION

The field of the present invention pertains to in vitro preparation of stem cells suitable for cell therapy.

### STATE OF THE ART

The use of stem cells represents an innovative tool for several tissue repair and reconstruction strategies as, for instance, in myocardial tissue repair after occurrence of myocardial infarction or as a result of congenital or acquired hypertrophic or dilated cardiomyopathies associated with destruction of cardiomyocytes, that represent the cardiac contractile units.

These new strategies are of extreme interest particularly in industrialized Countries where cardiovascular diseases represent a major cause of mortality. The attempts made so far have involved direct injection of undifferentiated stem cells in the cardiac tissue of mice or rats experimentally subjected to myocardial infarction (Orlic D, ET to. Nature. 2001; 410:701-705.). Most injected cells were eliminated by apoptotic phenomena and only a small percentage was “instructed” by the recipient myocardium to differentiate into embryonic cardiomyocytes through as yet unidentified mechanisms. By this strategy, transplanted stem cells must differentiate into cardiomyocytes in vivo, in the recipient's myocardium, resulting in very low yield, poor repair of tissue damage and minimal functional recovery from the hemodynamic point of view.

Instead, experiments performed in mouse models of heart failure have shown that cardiomyocytes already differentiated from totipotent stem cells, and selected from other cellular phenotypes by <<gene trapping>>, can survive after myocardial transplantation and integrate permanently in the myocardial tissue of the recipient (Klug M G et al J. Clin Invest. 1996; 98:216-224.).

However, the mechanisms underlying the induction of cell differentiation are very complex and only partially elucidated. Through the activation of a restricted number of tissue-specific transcription factors, these mechanisms orchestrate multiple profiles of gene expression directed towards specification of myocardial architecture.

The same authors of the present invention have contributed to illustrate some key steps of cardiogenesis, showing that expression of the genes encoding the transcription factors Gata-4 and Nkx-2.5 induces in embryonic stem cells the heart-specific transcripts α-myosin heavy chain (MHC) and myosin light chain 2-V (MLC) and a myocardial phenotype (Ventura C, Maioli M. Circ Res. 2000; 87:189-194). Moreover a complex autocrine endorphinergic system was identified that is capable of activating and orchestrating the expression of these “architect genes” of cardiogenesis (Luck C, Maioli M. Circ Res. 2000; 87:189-194).

In this context, processes that can precisely and efficiently induce stem cell differentiation are absolutely desirable and represent an innovative tool for several strategies of tissue repair and reconstruction.

In fact, the efficiency of the cardiogenic process in stem cells is, at present, strongly limited by the fact that differentiation towards the myocardial phenotype represents only a small part of all their possible differentiation fates. In pluripotent stem cells, that is to say endowed with more limited capacity to differentiate than totipotent stem cells of embryonic origin, the process of cardiogenesis can be induced by dimethylsulfoxide (DMSO). However, no more than 15-20% of these cells can differentiate in embryonic cardiomyocytes in presence of DMSO, as found in P19 cells, a pluripotent cell line derived from a mouse embryonic carcinoma (McBurney M W, Jones-Villeneuve E M W, Edwards M K S, Anderson P J. Nature. 1982,299:165-167). Moreover, DMSO is unable to produce a significant increase in the percentage of totipotent stem cells undergoing cardiomyocyte differentiation (Xu C, Police S, Rao N, Carpenter M K. Circ Res. 2002; 91:501-508). Retinoic acid, another substance that can act as stem cell differentiating agent, promotes various differentiation events that vary with the cell type under consideration, inducing neuronal differentiation in pluripotent P19 cells (McBurney M W, et al. Nature. 1982; 299:165-167) and cardiac differentiation in some stem cells lines (Wobus A M, et al. J Mol Cell Cardiol. 1997; 29:1525-1539) but lacking cardiomyocyte differentiation activity in human totipotent stem cells (Xu C, ET to Circ Res. 2002; 91:501-508).

Therefore, despite the fact that some cardiogenesis-determining mechanisms have being clarified, none of the currently available clinical and pharmacological treatments allows replacement of cardiomyocyte loss thereby arresting the progression of myocardial damage towards heart failure, that still represents a clinical irreversible event in a significant number of heart patients.

### SUMMARY

The present invention relates to the use of hyaluronic esters of retinoic acid as stem cell pro-differentiation agents, in particular, to their ability to promote the appearance of a myocardial phenotype characterized by the presence of embryonic cardiomyocytes endowed with spontaneous contractile activity.

The esters of the invention are partially or totally esterified with retinoic acid. When hyaluronic acid is not completely esterified with retinoic acid, it can be esterified with other short chain alkanoic acids, as for instance propanoic acid or butyric acid, the latter being preferred.

Moreover, the esters have preferably a molecular weight ranging between 10.000 and 30.000 Daltons: it is meant that such “molecular weight” refers to the average molecular weight (MW) of hyaluronic acid alone, without considering the contribution of butyric and retinoic residues.

Surprisingly, it has been observed that the pro-differentiating activity of retinoic acid esters on totipotent stem-cells is totally different from that of retinoic acid. In fact, these esters are able to differentiate stem cells towards cardiogenesis and to determine both a cardiogenic transcription profile (i.e. in the expression of genes that precede or induce the transcription of cardiospecific genes) and also the transcription of cardiospecific genes.

According to a further aspect, the invention also relates to the use of hyaluronic esters of retinoic acid to increase the efficiency of induction of the myocardial phenotype. Moreover, the invention relates to the use of these esters to generate cardiomyocytes that can be used in reconstructive cell therapy in all pathologies associated with destruction of cardiac contractile units.

In a further aspect, the invention relates also to the use of said esters for preparation of drugs that can be used in treatment and prevention of myocardial damage and in acute or congenital cardiomyopathies, in particular for treatment of myocardial infarction.

In a further aspect, the invention relates to a process for activation of genes responsible for cardiogenesis in mammalian stem cells, in particular murine and human cells, to the preparation and isolation of stem cells differentiated towards cardiogenesis, and to the preparation of in vitro cardiomyocytes.

Moreover, the invention extends to the use of isolated cells, differentiated according to the invention, to establish systems for in vitro selection of drugs for modulation of cardiogenesis.

Finally, the present invention includes a therapeutic method for treatment of heart failure caused by acquired pathologies (post-infarction, ischemic, or associated with valvular damage) or determined on genetic basis (hypertrophic or dilated cardiomyopathies), or due to myocardial infarction.

### DESCRIPTION OF THE FIGURES

FIG. 1. Effects of HRE on expression of genes that induce cardiogenic differentiation in embryonic stem cells.

After LIF withdrawal, cells were treated in absence (−) or presence (+) of retinoic esters of hyaluronic acid (0.75 mg/ml) for a total period of 10 days.

EBs: embryoid bodies 5 days after withdrawal of LIF (Leukemia Inhibitory Factor).

P: cardiomyocytes selected with puromycin 10 days after LIF withdrawal. A, B, C, RNase protection of Gata-4, Nkx-2.5 and prodynorphin mRNAs. On the left are shown autoradiography images relative to the expression of each transcript. On the right is shown the quantitative analysis of each mRNA level.

Meaningful differences relative to untreated cells.

FIG. 2. Effect of HRE on expression of cardiospecific genes in stem cells.

After LIF withdrawal, cells were treated in absence (−) or presence (+) of HRE-18 (0.75 mg/ml) for a total period of 10 days. The figure shows an ethidium bromide stained gel relative to the detection by RT-PCR of the effect of retinoic esters of hyaluronic acid on expression of “alpha myosin heavy chain” (MHC) and “myosin light chain-2V (MLC). This effect was detected in embryoid bodies (EBs) 5 days after LIF withdrawal and in puromycin-selected cardiomyocytes (P) 10 days after LIF withdrawal. In a group of experiments, stem cells have been treated for 10 days with retinoic esters of hyaluronic acid (0.75 mg/ml) in presence of 1 μM chelerithrine (Chel), a specific PKC inhibitor. In these samples, the analysis of gene expression was performed on puromycin selected cardiomyocytes 10 days after LIF withdrawal.

FIG. 3. Effect of HRE on transcription rates of Gata-4 and Nkx-2.5 genes.

A,B, autoradiography images relative to “nuclear run-off transcription” of Gata-4 and Nkx-2.5 genes respectively. After LIF withdrawal, cells were treated in absence (−) or presence (+) of retinoic esters of hyaluronic acid (0.75 mg/ml) for a total period of 10 days. EBs, embryoid bodies 5 days after LIF withdrawal. P, puromycin selected cardiomyocytes 10 days after LIF withdrawal. In a group of experiments, stem cells were treated for 10 days with retinoic esters of hyaluronic acid (0.75 mg/ml) in presence of 1 μM chelerithrine (Chel), a specific PKC inhibitor. Transcriptional analysis was then performed in nuclei isolated from puromycin selected cardiomyocytes 10 days after LIF withdrawal. a, transcription rates of Gata-4 or Nkx-2.5. b, transcription rate of the cyclophilin gene, used as internal control.

FIG. 4. Effect of HRE on transcription rate of the prodynorphin gene.

The figure shows the autoradiography image relative to “nuclear run-off transcription” of this gene. After LIF withdrawal, cells were treated in absence (−) or presence (+) of retinoic esters of hyaluronic acid (0.75 mg/ml) for a total period of 10 days. EBs, embryoid bodies 5 days after LIF withdrawal. P, puromycin-selected cardiomyocytes, 10 days after LIF withdrawal. In a group of experiments, the stem cells have been treated for 10 days with retinoic esters of hyaluronic acid (0.75 mg/ml) in presence of 1 μM chelerithrine (Chel), a specific PKC inhibitor. Transcriptional analysis was then performed in nuclei isolated from puromocin-selected cardiomyocytes 10 days after LIF withdrawal. a, transcription rate of the prodynorphin gene. b, transcription rate of the cyclophilin gene, used as internal control.

FIG. 5. Effect of HRE on the yield of the cardiogenic process in stem cells.

After LIF withdrawal (Day 0), GTR1 cells were cultured in absence (◯) or presence of retinoic esters of hyaluronic acid (0.75 mg/ml) (●) or of mixed esters of retinoic acid with hyaluronate and butyrate (♦) for a total period of 10 days. The number of cardiomyocyte colonies characterized by spontaneous contractile activity was considered as specification index of embryonic myocardial phenotype.

Significantly different from (◯); §, significantly different from (●).

### DETAILED DESCRIPTION OF THE INVENTION

The present invention relates to the use of hyaluronic esters of retinoic acid as stem cell pro-differentiation agents.

These esters affect the expression of cardiogenesis-inducing genes and of cardiospecific-genes in totipotent embryonic cells stem.

Moreover, they promote the appearance of a myocardial phenotype characterized by the presence of embryonic cardiomyocytes endowed with spontaneous contractile activity, preferably three-dimensionally organized.

The esters endowed with such activity are esters wherein the hydroxyl groups of the monosaccharidic units of hyaluronic acid are partially or totally esterified with retinoic acid. The degree of retinoic acid substitution in these esters preferably ranges from 0.00001 to 0.5 or, even more preferably, from 0.001 to 0.1, where the term degree of substitution (DS) indicates the number of moles of retinoic acid per mole of polysaccharide.

When hyaluronic acid is not totally esterified with retinoic acid, it can be esterified with other short chain alkanoic acids, as for example propanoic acid or butyric acid, the latter being preferred. In this specific case, the preferred mixed esters are mixed esters of hyaluronic acid with retinoic and butyric acids. Preferably, they have a degree of substitution with butyric acid ranging from 0.05 to 1.0, a degree of substitution with retinoic acid ranging from 0.002 to 0.1 and a ratio between substitution with butyric acid and retinoic acid (DS RAIDS BA) of at least 6.

For “degree of substitution” it is meant the number of esterifled hydroxyl groups for each repetitive unit of hyaluronic acid (dimer GIcNAc-GIcUA).

The term “retinoic acid” or “(RA)” designates all isomeric forms of this compound, therefore the natural form (with all the double bonds in trans), and all the other possible isomeric forms.

Moreover, the esters preferably have a molecular weight ranging from 10.000 to 30.000 Daltons: for “molecular weight” it is meant the average molecular weight (MW) of hyaluronic acid alone, without considering the contribution of butyric and retinoic residues.

Surprisingly it was observed that the retinoic acid esters have on totipotent stem cells a pro-differentiation ability that is different from that of retinoic acid in that they are able to differentiate stem cells towards cardiogenesis. In fact it is well known that retinoic acid induces instead neuronal differentiation in pluripotent P19 murine cells (McBurney M W, et al. Nature. 1982; 299:165-167) whereas does not show any effect on human stem cells (Xu C., et al. Circ. Res. 2002, 91:501-508).

A cardiogenic-effect is instead observed in vitro when pluripotent murine cells are treated with DMSO, although the overall efficiency of this process is rather low.

The administration of polysaccharidic esters of retinoic acid to stem cells in culture according to the new use described is able to determine both a cardiogenic transcription profile (genes whose expression precede or induce transcription of, cardiospecific genes) and also the transcription of cardiospecific genes.

The effects of the esters of the invention extend to all mammalian stem cells, preferably of embryonic origin, such as for example H1, H7, H9, H9.1 and H9.2 cells described in Thomson J A, et al. Science, 1998, 282:1145-1147 and in Amit M, et al. Dev Biol., 2000, 227:271-278, or to stem cells that can be isolated according to methods known in the art.

The effect of the esters of the invention has been measured in totipotent murine stem cells, preferably GTR1 (Nagy A, et al. Proc Natl Acad Sci USA. 1993; 90:8424-8428), selectable for a myocardial phenotype by “gene trapping” (Klug M G, et al. J Clin Invest 1996; 98:216-224). However, the “gene trapping” strategy represents an approach of phenotypic selection and not a tool that can increase the efficiency of the cardiogenic process. Therefore, the effect of the esters of the invention extends also to stem cells not selectable by “gene-trapping”.

The treatment with retinoic acid esters induces transcription of genes involved in cardiogenesis. Particularly increased are transcripts corresponding to prodynorphin, to the Nkx-2.5 homeodomain transcription factor (homologous to the Drosophila tinman gene), mutations of which determine congenital cardiomyopathies also in human, and to GATA-4 that encodes a protein belonging to the “zinc-finger domain” family. Moreover, transcription of the cardiospecific alpha-myosin heavy chain gene and of the gene for isoform 2V of myosin light chain (Mlc-2v) is increased. The latter is a particularly important marker because identifies ventricular positioning of cardiac myocytes during cardiogenesis. The observed effect cannot be simply ascribed to transcript stabilization, but is rather due to, an increase of the transcription rate of these genes. Therefore, the first aspect of the invention relates to the use of hyaluronic esters of retinoic acid to increase the efficiency of the process to induce cardiogenesis in undifferentiated stem cells. This is accomplished preferably by activation of cardiogenic genes such as prodynorphin, Nkx-2.5 homeodomain and GATA4 that belongs to the zinc-finger family.

The second aspect of the invention relates to the use of hyaluronic esters of retinoic acid to increase the transcription of cardiospecific genes as, for example, those encoding essential proteins in the process of cardiac muscle contraction, such as alpha-myosin heavy chain and myosin light chain 2V (MLC-2V).

A further aspect of the invention relates to the use of hyaluronic esters of retinoic acid to increase the overall efficiency of the process of myocardial phenotype induction, characterized by the generation from stem cells of embryonic cardiomyocytes endowed with spontaneous contractile activity.

The increased expression of cardiogenic and cardiospecific genes induced by retinoic esters of hyaluronic acid demonstrates that these substances are able to manipulate the genetic program of cardiac differentiation, offering a possible alternative to the complex and sometimes hazardous techniques of gene therapy. In an in vitro system of totipotent stem cells, the esters of the invention are particularly active at concentrations preferably ranging from 0.01 to 5 mg/ml, even more preferably ranging from 0.5 to 2.0 mg/ml.

Therefore, the esters of the present invention turn out to be extremely innovative compounds for reconstructive cell therapy in case of myocardial infarction, of hypertrophic or dilated cardiomyopathies determined on congenital or acquired bases, thus of all pathologies associated with destruction of the cardiac contractile units: the cardiomyocytes. In fact such cells are quiescent and, unlike skeletal muscle myocytes or other cell types such as cutaneous and hepatic cells, are unable to proliferate in response to tissue damage.

In a further aspect, the invention also relates to the use of the esters of the invention for preparation of drugs that can be used for treatment and prevention of myocardial damage and in acute or congenital cardiomyopathies, in particular for the treatment myocardial infarction.

In a further aspect, the invention relates to a process for activation of genes responsible for cardiogenesis in mammalian stem cells, in particular murine and human cells, and thus for preparation and isolation of stem cells differentiated towards cardiogenesis. The process essentially comprises a step of incubation of stem cells in a culture medium as, for example DMEM preferably in presence of serum, to which the esters are added in an amount ranging from 0.01 to 5 mg/ml, even more preferably ranging from 0.5 to 2.0 mg/ml. In this context, the increment of the yield of the cardiogenic process, as estimated from the number of cell colonies-characterized by spontaneous contractile activity, is at least 3-fold higher with respect to untreated control cells (FIG. 5). The treatment with esters is optionally followed by a step of cardiomyocyte selection, preferably by means of “gene-trapping” in which the chimeric gene consists of the “alpha-myosin heavy chain” gene promoter followed by a gene conferring resistance to chemotherapeutic agents that can be also different from puromycin as, for instance, geneticin or G418. Moreover, the chimeric gene used for selection by <<gene trapping>> could be stably integrated into the genome of the stem cell line, acs in the case of GTR1 cells, or could be only transiently introduced in stem cells, for instance by electroporation (Klug M G. et al J. Clin Invest. 1996; 98:216-224). In the latter case, the chimeric construct will not become integrated into the cellular genome but will be present in the nucleus where it can be transcribed for a limited time, although sufficient to operate the process of phenotypic selection. Although this “gene trapping” strategy does not result in a stable cell line for the selective process, it is of remarkable interest in view of a possible cell therapy approach to myocardial damage in human, where a time-limited permanence and expression of a chimeric gene in stem cells is desirable. The esters of the invention are also used in combination with selective approaches that are different from “gene trapping”, such as mechanical isolation of spontaneously contracting cardiomyocyte colonies, as for example by means of Pasteur pipette.

The efficiency of the overall cardiogenic process is measured, at the end of the selection, preferably as the percentage increment of the number of cardiomyocyte colonies with spontaneous contractile activity, with respect to the number of colonies appeared in not-treated controls.

Therefore, the cells differentiated according to the invention preferably further selected, consist of capable of contracting embryonic cardiomyocytes rather than of undifferentiated elements. Therefore, the cardiomyocytes obtained according to the description of the invention are useful in cell therapy of patients with myocardial infarction or heart failure caused by acquired pathologies (post-infarction, ischemic, or associated with valvular damage) or determined on a genetic basis (hypertrophic or dilated cardiomyopathies).

Furthermore, the invention extends to the use of the isolated cells, differentiated according to the invention, for setting up in vitro systems to select drugs for modulation of cardiogenesis, preferably for modulation of at least one of the following genes: GATA, preferably GATA-4, Nkx-2.5, alpha-myosin heavy chain, myosin light chain, prodynorphin, d-HAND, MEF and genes for cardiac ionic channels.

Alternatively, the differentiation process according to the invention can be used for the selection of molecules pharmacologically active in modulating heart development or activity or myocardial tissue repair processes. In these tests, stem cells are preferably chosen among: P19 cells, D3 cells, R1 cells, cells GTR1, H1, H7, H9, H9.1 and H9.2 cells described in Thomson J A, et al. Science, 1998, 282:1145-1147 and in Amit M, et al. Dev Biol., 2000, 227:271-278, or stem cells that can be isolated according to the methods known in the art.

The esters of the present invention are cardiogenic agents for stem cells and are therefore usable for repairing myocardial damage with autologous or heterologous stem cells. Therefore, the present invention includes a process to induce cardiogenic differentiation ex-vivo in autologous or heterologous stem cells for heart tissue repair in myocardial infarction or in heart failure caused by acquired pathologies (post-infarction, ischemic, or associated with valvular damage) or determined on a genetic basis (hypertrophic or dilated cardiomyopathies). This process includes the treatment of autologous or heterologous stem cells with the esters of the present invention in suitable culture medium, and optionally the selection of contractile cardiomyocytes and their subsequent in vivo re-implantation.

Therefore, the present invention includes a therapeutic method for treatment of heart failure caused by acquired pathologies (post-infarction, ischemic, or associated with valvular damage), or determined on a genetic basis (hypertrophic or dilated cardiomyopathies) or myocardial infarction. The method includes the isolation of stem cells preferably autologous, the treatment of said cells with retinoic esters of hyaluronic acid, and optionally the selection of differentiated stem cells for further re-implantation of the differentiated cardiomyocytes in the patient. The survival rate of cells that underwent in vitro or ex vivo differentiation into contractile embryonic cardiomyocytes is higher than that obtained in current pre-clinical studies that use undifferentiated cells. Moreover, because of their embryonic characteristics, grafted cardiomyocytes still possess proliferative activity and theoretically could give rise to a considerable myocardic mass before they further differentiate in adult cells and enter a stable phase of proliferative quiescence.

Therefore, the result obtained with the esters of the present invention is extremely useful because it demonstrates an increased efficiency of a process that activates a restricted number of tissue-specific transcription factors and to orchestrate multiple profiles of gene expression finalized to intra- and supra-cellular specification of a myocardial architecture. The results also show an increase of the overall efficiency of the cardiogenesis process finalized to the reproduction of myocardial architecture in vitro, that is least two-fold higher with respect to untreated cells.

Given the effect obtained with the esters of the present invention on coordination of the initial and specific events of cardiac differentiation, the use of said esters is therefore extended to the preparation of cardiomyocytes in vitro.

### EXPERIMENTAL PART

Methods

Analysis of gene expression. Total RNA has been extracted from stem cells as described by Ventura et al. (Ventura C et al. J Biol. Chem. 1997; 272: 6685-6692). The levels of specific mRNAs have been analyzed by “RNase protection”, using [³²P]CTP radiolabeled cRNA probes (antisense mRNA) specific for each transcript of interest, according to a previously described protocol (Ventura C et al. J Biol. Chem. 1997; 272:6685-6692). Briefly, fragments corresponding to the main exons of GATA-4 (292 bp), Nkx-2.5 (414 bp) and prodynorphin (424 bp) genes have been inserted in the vector pCRII-TOPO (Invitrdgen). Transcription of the vector linearized With ApaI, BamHI, or XbaI respectively generated sense strand mRNA of prodynorphin, GATA-4, or Nkx-2.5. Transcription in presence of [³²P]CTP of the plasmid linearized with BamHI produced the antisense mRNA strand of prodynorphin and Nkx-2.5, while transcription of the vector linearized with XbaI generated the antisense mRNA strand of GATA-4. In some experiments the analysis of mRNA expression has been performed by means of RT-PCR, as described in (Ventura C, Maioli M. Circ Res. 2000:87:189-194).

Gene transcription studies. Gene transcription rate has been analyzed in nuclei isolated by an approach of “in vitro nuclear run-off transcription”, previously described (Ventura C., J Biol Chem. 1997; 272:6685-6692.; Ventura C. et al. J Biol. Chem. 1998; 273:13383-13386.). Briefly, nuclei have been resuspended in buffer containing 50 mmol/L Tris/HCl, pH 8.0, 5 mmol/L MgCl₂, 0.1 mmol/L EDTA, 40% glycerol, 0.1 mmol/L dithiothreitol, 0.5 mmol/L phenylmethylsulfonylfluoride, 1 μmol/L leupeptin, and 10 mmol/L β-mercaptoethanol. 100 μl of 2× reaction buffer (10 mmol/L Tris/HCl, pH 7.5, 5 mmol/L MgCl₂, 0.3 mol/L KCl, 5 mmol/L dithiothreitol, 1 mmol/L of ATP, GTP, and CTP), and 5 μl of [α-³²P]UTP (3000 Ci/mmol) have been added to the nuclear suspension (90 μl). Samples were then incubated at room temperature for 15 min. The reaction has been stopped by digesting the DNA in presence of RNase-free DNase. The nuclear RNA has been isolated as described in Ventura C et al. J Biol Chem. 1997; 272:6685-6692. Approximately 5×10⁶ cpm of ³²P labeled nuclear RNA were hybridized for 12 hours at 55° C. in presence of cold (not radioactive) probes of antisense mRNA of GATA-4, Nkx-2.5 or prodynorphin, generated, as described above, in absence of radiolabeled ribonucleotides. Samples have been then incubated with a mixture of RNase A and T1, and exposed to proteinase K. The hybrid fragments have been recovered by phenol/chloroform extraction and separated by electrophoresis on non-denaturing polyacrylamide gel. The radiolabeled nuclear RNA has been also hybridized to the cyclophilin antisense mRNA synthesized from a pBS vector linearized with NcoI, containing a 270 bp fragment of rat cyclophilin clone pIB15.

The cyclophilin mRNA has been used as constant mRNA control.

### Example 1

### Induction of Cardiogenic and Cardiospecific Transcripts in GTR1 Stem Cells Treated with Esters of Hyaluronic Acid

GTR1 stem cells were used in these studies. This is a cell line derived from murine totipotent R1 cells (Nagy A, et al. Proc Natl Acad Sci USA. 1993; 90:8424-8428), containing a chimeric gene composed of the “alpha-myosin heavy chain” promoter followed by a gene capable of conferring resistance to puromycin (Ventura C. et al. J Biol. Chem. 1997; 272:6685-6692). The promoter of a cardiospecific gene (e.g. “alpha-myosin heavy chain”) “drives” the gene encoding a protein capable of conferring cellular resistance to a specific chemo-therapeutic agent. Cells can be maintained in an undifferentiated state in presence of “Leukemia Inhibitory Factor (LIF)”. The substance was present at a final concentration of 1000 U/ml. The culture medium was DMEM, containing 15% fetal bovine serum (FBS). In order to induce differentiation, it is necessary to remove the LIF. Therefore cells have been cultured, without LIF, in DMEM containing 15% FBS in Petri dishes for bacterial culture. After 2 days, the resulting embryoid bodies (EBs) have been transferred in dishes capable of inducing cell adhesion (“tissue culture dishes”). After 3-5 days, at the appearance of the first cell contractions, indicating the onset of the cardiogenic commitment of part of the cells in culture, puromycin is added at a final concentration of 2 μg/ml. In addition to activating the native promoter, the cells oriented towards cardiogenesis will activate the promoter of the chimeric gene, thereby making possible to separate them from the remaining “non myocardial” cells-since they become resistant to the chemotherapeutic agent. In this way, it is possible to select, by an innovative “gene trapping” approach, only cells undergoing the process of cardiogenesis.

After LIF withdrawal, and during puromycin selection, GTR1 cells were exposed to the various retinoic esters of hyaluronic acid. The EBs, collected in various steps of the cardiogenic process, and the puromycin-selected cardiomyocytes, have been processed for analysis of the specific gene expression profiles correlated with induction of cardiogenesis or with the established commitment to a myocardial phenotype. At last, the number of contractile colonies was quantified as indicator of the yield of the cardiac differentiation process.

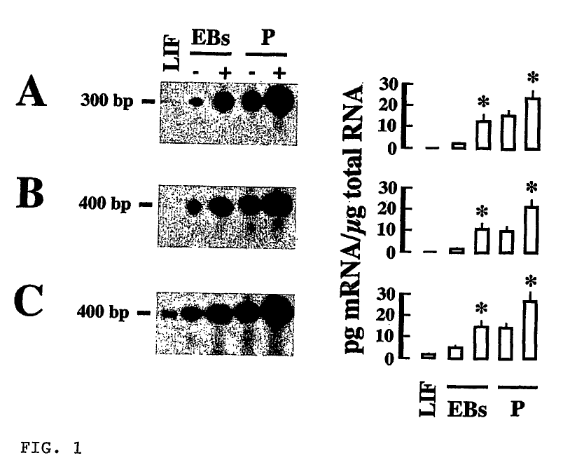

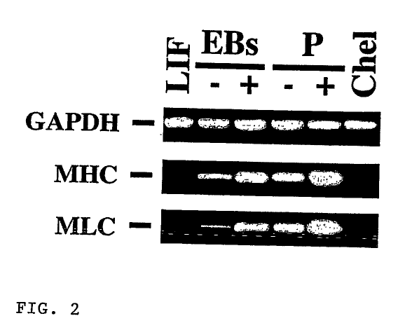

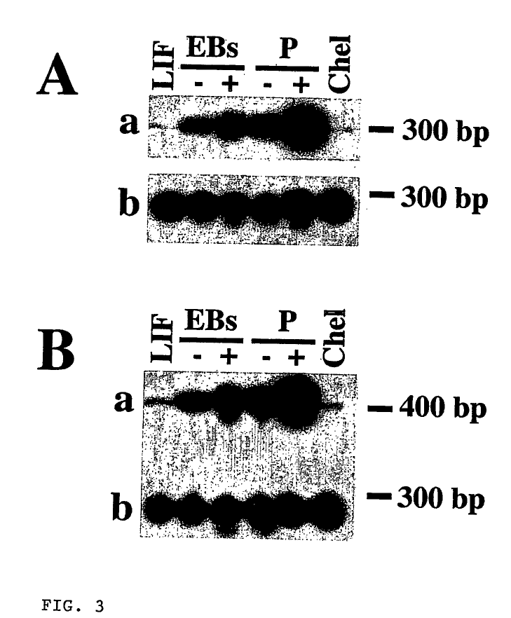

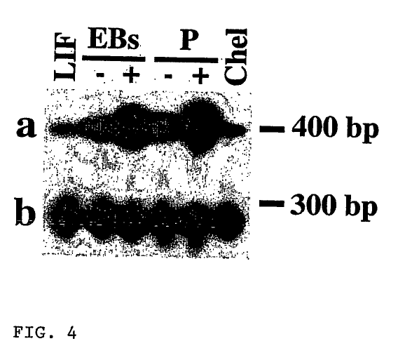

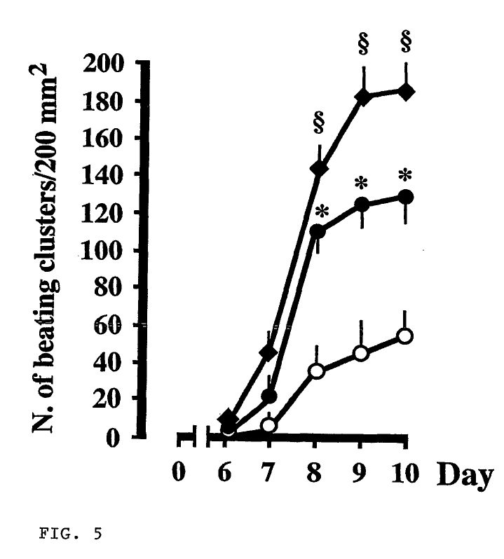

FIG. 1 shows a quantitative analysis, carried out by “RNase protection”, of the expression of some cardiogenesis-inducing genes in GTR1 cells treated in absence and in presence of retinoic esters of hyaluronic acid, after LIF withdrawal. It is evident that retinoic esters of hyaluronic acid were capable of inducing a remarkable increase of GATA4 and Nkx-2.5 mRNA levels in the embryold bodies collected 5 days after LIF withdrawal and in puromycin-selected cardiomyocytes. The first of these transcripts encodes for a transcription factor of the “zinc finger” family, while the second directs the expression of a “homeodomain” protein. Both transcription factors play a crucial role in embryonic cardiac differentiation in several animal species, including man (Lints T J, et al. Development, 1993; 119:419-431. Schott J J et al. Science. 1998; 281:108-111. Benson D W, J Clin Invest 1999; 104:1567-1573). Moreover, mutations in these genes have been recently found to be associated with severe abnormalities of cardiac development not compatible with neonatal survival (Schoft J J, et al Science. 1998; 281:108-111. Benson D W, et al. J Clin Invest. 1999; 104:1567-1573). FIG. 1 shows that the substance object of the invention also produced a marked increase of the expression of the prodynorphin gene, whose peptide products turned out to trigger and orchestrate the expression of GATA-4 and Nkx-2.5 genes in stem cells (Ventura C, Maioli M. Circ Res. 2000; 87:189-194). The other HREs (HRE-15 AND HRE-16) produced similar results. Moreover all the HREs tested have produced a remarkable increase of the expression of cardiospecific transcripts coding “alpha-myosin heavy chain” (MHC) and “myosin light chain-2V” (MLC) (FIG. 2). No significant change of GATA-4 and NKx-2.5 gene expression was detected after exposure of GTR1 cells to hyaluronic acid (0.75 mg/ml).

### Example 2

### Measurement of the Transcription Rate of Cardiospecific Genes in Stem Cells

“Nuclear run-off” experiments performed with nuclei from cells treated with retinoic esters of hyaluronic acid showed that the effects on GATA-4 and Nkx-2.5 gene expression (FIG. 3) and on prodynorphin gene expression (FIG. 4) could be attributed to an effective increase of transcription rate. These results rule out a mere effect of the substance at the level of mRNA stability. The effects produced by the retinoic esters of hyaluronic acid on expression of cardiogenic and cardiospecific genes have been antagonized by the presence in the culture medium of chelerithrine, a specific protein kinase C (PKC) inhibitor (FIGS. 2-4). This result suggests the involvement of a PKC-dependent signal transduction pathway in the cardiogenic effect produced by the substances under study.

### Example 3

### Comparative Analysis of the Entire Cardiogenic Process

FIG. 5 shows a comparative analysis of the yield of cardiogenic process in GTR1 cells cultured in absence and in presence of retinoic esters of hyaluronic acid. Cells were cultured in absence (empty circle) or presence (filled circle) of retinoic esters of hyaluronic acid (0.75 mg/ml) for a total period of 10 days. The number of cardiomyocyte colonies characterized by spontaneous contractile activity has been considered as index of the specification of an embryonic myocardial phenotype.

It is evident that the retinoic esters of hyaluronic acid are capable of producing a marked increase of the number of cardiomyocyte colonies endowed with contractile activity, that is a phenotypic marker of accomplished cardiogenesis.

### Example 4

### Treatment of Totipotent Stem Cells with Mixed Esters of Retinoic Acid

In a separate series of experiments, GTR1 cells have been exposed to the action of mixed esters of retinoic acid with hyaluronic acid and butyrate (0.75 mg/ml). After 8 days it was possible to observe a significant increase of contractile colonies as result of treatment with these substances, not only with respect to untreated control plates but also with respect to the plates treated with individual retinoate-hyaluronate esters (FIG. 5). These effects were accompanied by activation of a cardiogenic gene program comprising the induction of GATA-4 and Nkx-2.5 genes and increased expression of the prodynorphin gene.

In conclusion, the obtained results demonstrate that retinoic esters of hyaluronic acid and mixed esters of retinoic acid with hyaluronate and butyrate behave as powerful morphogenetic agents capable of inducing and orchestrating the process of cardiogenesis in totipotent embryonic stem cells.

1. Use of retinoic esters of hyaluronic acid as stem cells pro-differentiating agents.

2. Use according to claim 1, wherein such esters are characterized in that they have a degree of substitution with retinoic acid is comprised from 0.00001 to 0.5.

3. Use according to claim 2, wherein said degree of substitution with retinoic acid is comprised from 0,002 to 0.1.

4. Use according to claim 1, wherein such esters are mixed esters of hyaluronic acid with butyric and retinoic acids.

5. Use according to claim 4, wherein the mixed esters are characterized in that they have degree of substitution with butyric acid ranging from 0.05 to 1.0, a degree of substitution with retinoic acid ranging from 0.002 to 0.1 and a ratio between the degree of substitution with butyric acid and retinoic acid (DS RA/DS BA) of at least 6.

6. Use according to claim 1, wherein said stem cells are mammalian.

7. Use according to claim 6, wherein said mammalian are chosen among: H. sapiens, primates, higher primates, rodents, swine, bovines.

8. Use according to claims 1-7, wherein said stem cells are of embryonic or somatic origin.

9. Use of polysaccharidic esters of retinoic acid for preparation of medicaments with cardiogenic pro-differentiating activity on stem cells.

10. Use according to claim 9 for preparation of medicaments with a cardiogenic pro-differentiating activity.

11. Use according to claim 10 for preparation of drugs for treatment and prevention of myocardial damages and of cardiomyopathies

12. Use according to claim 11, wherein the myocardial damage is myocardial infarction.

13. Process for in vitro preparation of cardiomyocytes essentially comprising a step of incubation of stem cells with retinoic esters of hyaluronic acid and optionally a selection of the contractile units comprising said cardiomyocytes.

14. Process according to claim 13, wherein said retinoic esters are characterized by a substitution degree of hyaluronic acid with retinoic acid comprised from 0.00001 to 0.5.

15. Process according to claim 13, wherein such retinoic esters are mixed esters of hyaluronic acid with butyric and retinoic acids.

16. Process according to claim 15, wherein such mixed esters are characterized in that they have a degree of substitution with butyric acid ranging from 0.05 to 1.0, a degree of substitution with retinoic acid ranging from 0.002 to 0.1 and a ratio between the degree of substitution with butyric acid and retinoic acid (DS RA/DS BA) of at least 6.

17. Process according to claim 13, wherein said stem cells are autologous or heterologous.

18. Process according to claim 17, wherein the selection is performed by means of “gene-trapping”.

19. Process according to claim 17, wherein said stem cells are chosen among: P19, D3 cells, R1 cells, GTR1 cells.

20. Process for the selection of new molecules with cardiogenic-modulation activity comprising the process according to claims 13-19 and optionally a step for optimization of the selected molecules.

21. Process for preparation of an in vitro cell model for cardiogenic differentiation of stem cells, essentially comprising a step of incubation of said stem cells with retinoic esters of hyaluronic acid alone or in combination with other substances, in suitable culture medium.

22. Process according to claim 21, wherein such retinoic esters are characterized in that they have a degree of substitution of hyaluronic acid with retinoic acid ranging from 0.00001 to 0.5.

23. Process according to claim 22, wherein such retinoic esters are mixed esters of hyaluronic acid with butyric and retinoic acids.

24. Process according to claim 23, wherein such mixed esters are characterized in that they have a degree of substitution with butyric acid ranging from 0.05 to 1.0, a degree of substitution with retinoic acid ranging from 0.002 to 0.1° and a ratio between the degree of substitution with butyric acid and with retinoic acid (DS RAIDS BA) of at least 6.

25. Process according to claim 21, wherein said stem cells are chosen among: P19, D3, R1, GTR1, H1, H7, H9, H9.1 and H9.2 cells.

26. Process according to claim 21, wherein such incubation is followed by a step of selection of the contractile units comprising cells differentiated in cardiomyocytes.

27. A therapeutic method for treating heart failure in a patient in need of such a treatment characterised in that heterologous or autologous stem cells are treated “in vitro” or “ex vivo” with retinoic esters of hyaluronic acid.

28. A therapeutic method according to claim 27 wherein the degree of substitution of hyaluronic acid with retinoic acid is comprised from 0,00001 to 0,5.
